(19) United States
(12) Patent Application Publication
(10) Pub. No.: US 2023/0277722 A1
(43) Pub. Date: Sep. 7, 2023

(54) DRY DRESSING

(71) Applicant: CYMMETRIK ENTERPRISE CO.,LTD., Taipei City (TW)

(72) Inventor: Yu-Ju LIU, Taipei (TW)

(73) Assignee: CYMMETRIK ENTERPRISE CO.,LTD., Taipei City (TW)

(21) Appl. No.: 18/114,973

(22) Filed: Feb. 27, 2023

(30) Foreign Application Priority Data
Mar. 4, 2022 (TW) 111107863

(51) Int. Cl. A61L 26/00

(52) U.S. Cl. CPC A61L 26/0052; A61L 26/0066; A61L 26/008; A61L 2300/102; A61L 2300/252; A61L 2300/214

(57) ABSTRACT

This disclosure relates to a dry dressing configured to be applied on human skin. The dry dressing includes a substrate layer and a contact layer that is disposed on the substrate layer. The contact layer includes a hydrocolloid dryly formed on the substrate layer and a nutrient mixed in the hydrocolloid. The hydrocolloid includes polyvinyl alcohol and polyvinylpyrrolidone. A weight percentage of the polyvinyl alcohol and the polyvinylpyrrolidone to the hydrocolloid is not less than 96%, and a weight ratio of the polyvinyl alcohol to the polyvinylpyrrolidone ranges from 0.725 to 0.785.

### CROSS-REFERENCE TO RELATED APPLICATIONS

This non-provisional application claims priority under 35 U.S.C. § 119(a) on Patent Application No(s). 111107863 filed in Taiwan, R.O.C. on Mar. 4, 2022, the entire contents of which are hereby incorporated by reference.

### TECHNICAL FIELD

The present disclosure relates to a dressing, more particularly to a dry dressing having a dried hydrocolloid.

### BACKGROUND

Dressings for medical or cosmetic purpose are used to attach to human skin to achieve the skin treating or caring function. Some types of nutrients need to be carried by liquid for better absorption, thus a wet dressing is to soak dressing material in liquid substance and sealed in a bag or container.

Typically, wet dressings contain preservatives to stop germs, but preservatives may cause an allergic reaction to some people's skin. Also, the wet dressing has a certain weight. Moreover, user needs to carefully store the dressing package to prevent pressure from breaking it. Once the container seal is broken and open to air, it is no longer considered sterile and the liquid is tendency to flow out, thereby making the dressing unusable.

### SUMMARY

The present disclosure provides a dry dressing which is easy to carry and store and has no irritation or allergic reaction to human skin.

According to one aspect of the present disclosure, a dry dressing configured to be applied on human skin. The dry dressing includes a substrate layer and a contact layer that is disposed on the substrate layer. The contact layer includes a hydrocolloid dryly formed on the substrate layer and a nutrient mixed in the hydrocolloid. The hydrocolloid includes polyvinyl alcohol and polyvinylpyrrolidone. A weight percentage of the polyvinyl alcohol and the polyvinylpyrrolidone to the hydrocolloid is not less than 96%, and a weight ratio of the polyvinyl alcohol to the polyvinylpyrrolidone ranges from 0.725 to 0.785

According to the dry dressing discussed above, the hydrocolloid and the nutrient mixed therein dryly formed on the substrate layer helps the dry dressing to be light enough and to be carried and stored easily. Also, additional preservatives is prevented to be added into the dry dressing, and therefore the dry dressing has no irritation or allergic reaction to human skin.

Moreover, by designing the weight ratio of the PVA to the PVP ranging from 0.725 to 0.785, the hydrocolloid is able to fully absorb the water within 3 seconds, and the dry dressing has a proper weight and a proper fixity on human skin for releasing the nutrient.

### BRIEF DESCRIPTION OF THE DRAWINGS

The present disclosure will become more fully understood from the detailed description given hereinbelow and the accompanying drawings which are given by way of illustration only and thus are not intending to limit the present disclosure and wherein:

FIG. 1 is a side view of a dry dressing according to one embodiment of the present disclosure;

FIG. 2 to FIG. 3 are schematic views showing the manufacturing process of the dry dressing in FIG. 1; and

FIG. 4 is a schematic view showing how to use the dry dressing in FIG. 1.

### DETAILED DESCRIPTION

Aspects and advantages of the invention will become apparent from the following detailed descriptions with the accompanying drawings. For purposes of explanation, one or more specific embodiments are given to provide a thorough understanding of the invention, and which are described in sufficient detail to enable one skilled in the art to practice the described embodiments. It should be understood that the following descriptions are not intended to limit the embodiments to one specific embodiment. On the contrary, it is intended to cover alternatives, modifications, and equivalents as can be included within the spirit and scope of the described embodiments as defined by the appended claims.

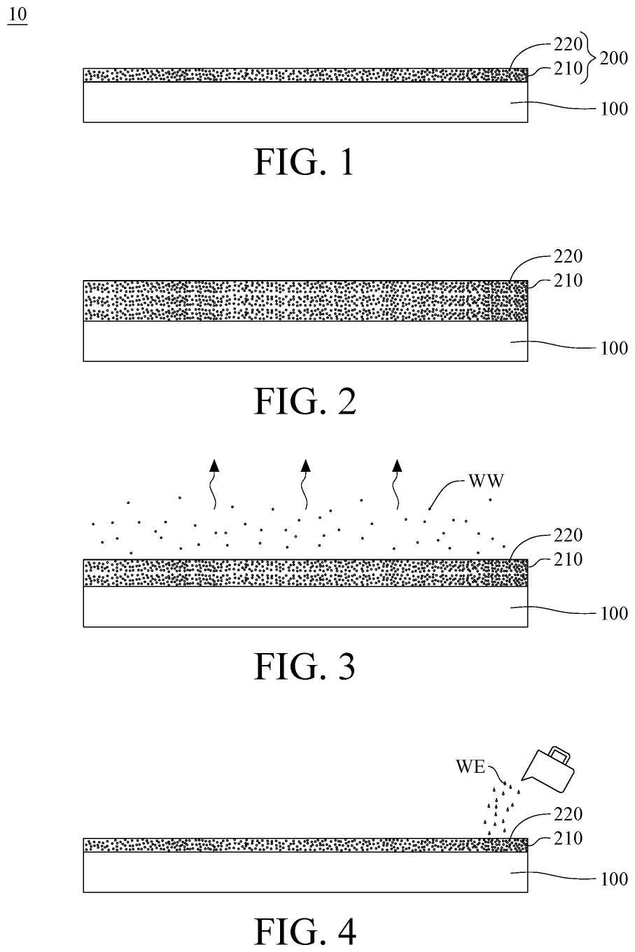

One embodiment of the present disclosure will be illustrated hereinafter with reference to FIG. 1 to FIG. 3, where FIG. 1 is a side view of a dry dressing 10 according to one embodiment of the present disclosure, and FIG. 2 to FIG. 3 are schematic views showing the manufacturing process of the dry dressing 10.

As shown, the dry dressing 10 includes a substrate layer 100 and a contact layer 200. The contact layer 200 is disposed on the substrate layer 100, and the contact layer 200 includes a hydrocolloid 210 and a nutrient 220. As shown, the hydrocolloid 210 and the nutrient 220 are mixed to each other and distributed in the same layer.

Specifically, the nutrient 220 is in a wet state to mix with the hydrocolloid 210, thus the nutrient 220 is able to be uniformly distributed in the hydrocolloid 210. In some embodiments, the nutrient may be mixed with the hydrocolloid when in a dry state. The contact layer 200 including the hydrocolloid 210 and the nutrient 220 mixed therein, as shown in FIG. 2, may be applied to the substrate layer 100 using, for example, screen printing, coating, dispensing, jet printing or impregnating. The substrate layer 100 and the contact layer 200 may be placed in a dry environment to dry out the hydrocolloid 210. As shown in FIG. 3, more and more water WW come out and therefore the hydrocolloid 210 becomes dry on the substrate layer 100.

The dry dressing 10 is suitable to contact human skin after getting wet. Please further refer to FIG. 4 to describe how to use the dry dressing 10. As shown, water or essential oil WE is provided to the dry dressing 10. The hydrocolloid 210 is able to absorb the water or essential oil WE and therefore gets wet and expands. After a short period of time, the hydrocolloid 210 may reach a high osmotic pressure, such that hydrophilic cross-linked polymer inside the hydrocolloid 210 increases the fluid pressure therein to push the nutrient 220 outward. Therefore, when the dry dressing 10 gets wet and is attached to human skin, the nutrient 220 tends to go into human skin. In an experiment performed on the dry dressing 10 with about 4 to 5 grams of hydrocolloid 210: the hydrocolloid 210 is able to fully absorb about 7.5 grams of water after 30 seconds; when 15 grams of water is added to the dry dressing 10, the hydrocolloid 210 absorbs part of it and remain about 3 to 4 grams of water on the surface; when 22.5 grams of water is added to the dry dressing 10, the hydrocolloid 210 absorbs part of it and remain about 6 to 7 grams of water on the surface. The experiment shows that every unit of the hydrocolloid 210 may absorb a fixed amount of water and does not absorb water excessively.

The hydrocolloid 210 may include polyvinyl alcohol (PVA) and polyvinylpyrrolidone (PVP), wherein the PVA is provided to enhance the strength of the hydrocolloid 210, and the PVP is provided to enhance the hydrophile of the hydrocolloid 210. In detail, in the hydrocolloid 210, the weight percentage of the PVA and the PVP to the hydrocolloid 210 is 96% or more, and the weight ratio of the PVA to the PVP in the hydrocolloid 210 may range from 0.725 to 0.785. As such, a proper balance between the formability and the hydrophile of the hydrocolloid 210 is obtained. As a result, when the dry dressing 10 gets wet, the hydrocolloid 210 is able to maintain a certain level of fluidity while keeping the nutrient 220 therein.

The following table shows seven formulas suitable for the dry dressing. Among them, the hydrocolloid is about 5 grams, the weigh percentage of the PVA and the PVP to the hydrocolloid is about 96%, and the nutrient contains phycoerythrin to reduce iodine solution. It is noted that the phycoerythrin is provided for user to determine how fast the dry dressing releases the nutrient after getting wet; specifically, the dry dressing can be soaked in iodine solution after gets wet, and then the nutrient 220 will release the phycoerythrin to reduce the iodine solution and thereby decolorize to the iodine solution. By doing so, it is possible to determine the level of releasing the nutrient by observing the transparency of the iodine solution.

| | | | | Whether the |
|---|---|---|---|---|
| | | | | releasing of |
| | | | | the nutrient |
| | | | | is obvious |
| | | | | (observing the |
| | Weight | | | interaction of |
| | ratio | | | phycoerythrin |
| | of PVA | Adding 22.5 | State after | and iodine |
| | to PVP | grams of water | getting wet | solution) |
| Formula 1 | 0.70 | Water is fully | Easy to spread, | Yes |
| | | absorbed in 2 | but it is too | |
| | | seconds | heavy and | |
| | | | easily falls out | |
| | | | of the user's | |
| | | | face | |
| Formula 2 | 0.72 | Part of water is | Easy to spread, | Yes |
| | | absorbed in 2 | but it is too | |
| | | seconds, and 3 | heavy and | |
| | | grams of water | easily falls out | |
| | | remains | of the user's | |
| | | | face | |
| Formula 3 | 0.725 | Part of water is | Easy to spread, | Yes |
| | | absorbed in 2 | and having | |
| | | seconds, and 6 | proper weight | |
| | | grams of water | and proper | |
| | | remains | fixity | |
| Formula 4 | 0.75 | Part of water is | Easy to spread, | Yes |
| | | absorbed in 2 | and having | |
| | | seconds, and 6.5 | proper weight | |
| | | grams of water | and proper | |
| | | remains | fixity | |
| Formula 5 | 0.785 | Part of water is | Easy to spread, | Yes |
| | | absorbed in 3 | and having | |
| | | seconds, and 7 | proper weight | |
| | | grams of water | and proper | |
| | | remains | fixity | |
| Formula 6 | 0.79 | Part of water is | Difficult to | No |
| | | absorbed in 7 | spread | |
| | | seconds, and 10.5 | | |
| | | grams of water | | |
| | | remains | | |
| Formula 7 | 0.81 | Part of water is | Difficult to | No |
| | | absorbed in 13 | spread, and | |
| | | seconds, and 16 | uncomfortably | |
| | | grams of water | warm and wet | |
| | | remains | | |

As can be seen from the formulas 3-5, the hydrocolloid 210 is able to fully absorb the water within 3 seconds when the weight ratio of the PVA to the PVP ranges from 0.725 to 0.785, thus the dry dressings 10 in formulas 3-5 are suitable to be used with water or essential oil. As can be seen from the formulas 1-2, they need absorb more water to reach the required expansion and their hydrocolloids become fluidly when the weight ratio of the PVA to the PVP is less than 0.725 (i.e., an overly high proportion of the PVP), as a result, the weight of theses dry dressings is quite noticeable by the user and the hydrocolloids are unable to be firmly fixed on the substrate layer, thereby causing user an uncomfortable feeling. As can be seen from the formulas 6-7, they need more time to reach the required expansion and their hydrocolloids tend to become too solid when the weight ratio of the PVA to the PVP is greater than 0.785 (i.e., an overly high proportion of the PVA), as a result, the dry dressings are not easily to be spread and would make user feel uncomfortably warm and wet. Moreover, regarding the formulas 6-7, the weight ratio of the PVA to the PVP greater than 0.785 is not beneficial for the releasing of the nutrient 220 since there is no obvious discoloration of the iodine solution.

It is noted that the hydrocolloid 210 may not only contain PVA and PVP. In some embodiments, the hydrocolloid 210 may include one or more other substances for achieving other effects. For example, the hydrocolloid 210 may further include sodium polyacrylate (PAA-Na). A small amount of PAA-Na can increase the viscosity of the hydrocolloid 210, thus the adding of the PAA-Na to the hydrocolloid 210 may enhance the tactile sensation while using the wetted dry dressing 10. In some embodiments, the weight percentage of the PAA-Na to the hydrocolloid 210 may range from 0.2% to 0.4%.

Optionally, the hydrocolloid 210 may further include xanthan gum and guaran (or called jaguar in some cases) to further increase the strength of the expanded hydrocolloid 210. The xanthan gum and the guaran therefore can prevent the hydrocolloid 210 from being overly supple when the PVA and the PVP in the hydrocolloid 210 encounter specific ions or a substance with an overly low or overly high pH value. The xanthan gum and the guaran can also facilitate the water-absorption of the hydrocolloid 210 and enhance the slippery feeling of the expanded hydrocolloid 210. In detail, the weight percentage of the xanthan gum to the hydrocolloid 210 may range from 0.4% to 0.5%, and the weight percentage of the guaran to the hydrocolloid 210 may range from 0.05% to 0.2%.

The following table shows three formulas of the PAA-Na, the xanthan gum, and the guaran as additions to the dry dressings, and the dry dressings are also evaluated by six evaluators. The result is evaluated sequentially by the tactile quality, the slippery feeling, and the plump feeling. The evaluation results are, from high to low, Formula Y, Formula X, and Formula Z, wherein the evaluation result of Formula Z is too low to be determined as unqualified.

| | Weight percentage | | | | Evaluation | | |
|---|---|---|---|---|---|---|---|
| | | Xanthan | | | Tactile | Slippery | Plump |
| | PAA-Na | gum | Guaran | Evaluator | quality | feeling | feeling |
| Formula | 0.2% | 0.45% | 0.08% | A | Good | No | Yes |
| X | | | | B | Good | No | Yes |
| | | | | C | Good | No | Yes |
| | | | | D | Good | No | Yes |
| | | | | E | Good | No | Yes |
| | | | | F | Good | No | Yes |
| Formula | 0.4% | 0.47% | 0.179% | A | Good | Yes | Yes |
| Y | | | | B | Good | Yes | Yes |
| | | | | C | Good | Yes | Yes |
| | | | | D | Good | Yes | Yes |
| | | | | E | Good | Yes | Yes |
| | | | | F | Good | Yes | Yes |
| Formula | 0.53% | 0.38% | 0.08% | A | Sticky | No | A little |
| Z | | | | B | Sticky | No | A little |
| | | | | C | Good | No | A little |
| | | | | D | Sticky | No | A little |
| | | | | E | Sticky | No | A little |
| | | | | F | Sticky | No | A little |

Optionally, the hydrocolloid 210 may further include carboxymethyl cellulose (CMC). In detail, the weight percentage of the CMC to the hydrocolloid 210 may be 0.3% or more. The existence of the carboxymethyl cellulose makes the hydrocolloid 210 possible to be dried within 6 minutes at a relatively low temperature (e.g., below 50° C.). This can prevent deterioration of the nutrient 220 due to high temperature (e.g., more than 50° C.). In some embodiments, the weight percentage of the CMC to the hydrocolloid 210 may range from 0.3% to 2.0% and therefore is beneficial to maintain a high weight percentage of the PVA and the PVP while having a proper drying time of the hydrocolloid 210. Further, designing the weight percentage of the CMC within the range from 0.3% to 2.0% can also save manufacturing cost. The following table shows the relationship between the weight percentage of the CMC to the hydrocolloid 210 and the required drying time of the hydrocolloid 210.

Weight percentage of CMC

| | 0.0% | 0.3% | 1.5% | 2.0% |
|---|---|---|---|---|
| Drying time | 8~10 | 5~6 | 3~3.5 | 3 |
| (minute) | | | | |

Optionally, the nutrient 220 may include pH-neutral substances, such as hyaluronic acid, fermented β-glucan, and extract of proteoglycan, active ingredients, such as protein and amino acid, antioxidants, such as phycoerythrin, or metal ions, such as calcium ions, magnesium ions, sodium ions, and potassium ions. Among them, the phycoerythrin is able to perform a reduce reaction, as discussed above, to reduce brown iodine molecules into colorless iodine ions. This can know that the phycoerythrin is able to protect skin and eliminate free radicals.

According to the dry dressing discussed above, the hydrocolloid and the nutrient mixed therein dryly formed on the substrate layer helps the dry dressing to be light enough and to be carried and stored easily. Also, additional preservatives is prevented to be added into the dry dressing, and therefore the dry dressing has no irritation or allergic reaction to human skin.

Moreover, by designing the weight ratio of the PVA to the PVP ranging from 0.725 to 0.785, the hydrocolloid is able to fully absorb the water within 3 seconds, and the dry dressing has a proper weight and a proper fixity on human skin for releasing the nutrient.

According to the dry dressing of the present disclosure, the hydrocolloid containing PVA and PVP is suitable for mixing pH-neutral ingredients as the nutrient therein, such as hyaluronic acid, fermented β-glucan, and extract of proteoglycan.

According to the dry dressing of the present disclosure, the hydrocolloid containing xanthan gum and guaran is suitable for mixing metal ions as the nutrient therein, such as calcium ions, magnesium ions, sodium ions, and potassium ions, or active ingredients that are poorly compatible with alcohol, such as protein or amino acid.

The embodiments are chosen and described in order to best explain the principles of the present disclosure and its practical applications, to thereby enable others skilled in the art best utilize the present disclosure and various embodiments with various modifications as are suited to the particular use being contemplated. It is intended that the scope of the present disclosure is defined by the following claims and their equivalents.

What is claimed is:

1. A dry dressing, comprising:
a substrate layer; and
a contact layer disposed on the substrate layer, wherein the contact layer comprises a hydrocolloid dryly formed on the substrate layer and a nutrient mixed in the hydrocolloid;
wherein the hydrocolloid comprises a polyvinyl alcohol and a polyvinylpyrrolidone, a weight percentage of the polyvinyl alcohol and the polyvinylpyrrolidone to the hydrocolloid is not less than 96%, and a weight ratio of the polyvinyl alcohol to the polyvinylpyrrolidone ranges from 0.725 to 0.785.

2. The dry dressing according to claim 1, wherein the contact layer is applied on the substrate layer by screen printing, coating, dispensing, jet printing or impregnating.

3. The dry dressing according to claim 1, wherein the hydrocolloid further comprises a sodium polyacrylate.

4. The dry dressing according to claim 3, wherein a weight percentage of the sodium polyacrylate to the hydrocolloid ranges from 0.2% to 0.4%.

5. The dry dressing according to claim 1, wherein the hydrocolloid further comprises a xanthan gum and a guaran.

6. The dry dressing according to claim 5, wherein a weight percentage of the xanthan gum to the hydrocolloid ranges from 0.4% to 0.5%, and a weight percentage of the guaran to the hydrocolloid ranges from 0.05% to 0.2%.

7. The dry dressing a cording to claim 5, wherein the nutrient comprises a metal ion.

8. The dry dressing according to claim 1, wherein the hydrocolloid further comprises a carboxymethyl cellulose.

9. The dry dressing according to claim 8, wherein a weight percentage of the carboxymethyl cellulose to the hydrocolloid is not less than 0.3%.

10. The dry dressing according to claim 1, wherein the nutrient comprises a protein or an amino acid.
